(19) United States
(12) Patent Application Publication
(10) Pub. No.: US 2006/0257447 A1
(43) Pub. Date: Nov. 16, 2006

(54) Composite graft

(75) Inventors: Monica Hinds, Portland, OR (US); David Courtman, Pickering (CA); Rebecca C. Rowe, Portland, OR (US)

Correspondence Address: KLARQUIST SPARKMAN, LLP, 121 SW SALMON STREET, SUITE 1600, PORTLAND, OR, 97204, US

(73) Assignee: Providence Health System

(21) Appl. No.: 11/371,531

(22) Filed: Mar. 8, 2006

Related U.S. Application Data
(60) Provisional application No. 60/660,536, filed on Mar. 9, 2005.

(51) Int. Cl. A61K 35/34

(52) U.S. Cl. 424/423; 424/569

(57) ABSTRACT

Composite vascular grafts, their methods of construction, and methods of use are disclosed. In some embodiments, the grafts are tubular multilayer composite grafts having a luminal blood-contacting elastin layer and an outer collagen layer to impart mechanical strength to the elastin layer. The elastin and collagen layers may comprise substantially acelluar matrixes isolated from the tissue of an animal. In particular embodiments the blood contacting layer consists essentially of pure elastin, and is substantially free of collagen. An adhesive may be used to secure the collagen and elastin layers to one another. Growth factors may be added to the adhesive of the elastin or collagen layers to promote in growth of cells into the graft.

### CROSS REFERENCE TO RELATED APPLICATION

This application claims priority of, and incorporates by reference, U.S. Provisional Patent Application No. 60/660,536, filed Mar. 9, 2005.

### STATEMENT OF GOVERNMENT SUPPORT

This invention was made with United States Government support under grant number DAMD 17-98-1-8654 from the United States Army. The United States Government has certain rights in the invention.

### FIELD

The present disclosure relates to biomaterials, such as biologically based biomaterials, for example, bioengineered prosthetic tissue that is suitable for use as a vascular graft. Methods are also disclosed for the preparation and use of the graft.

### BACKGROUND

Vascular damage and disease is a widespread problem encountered in clinical medicine. Modern dietary practices, for example, have produced a high prevalence of cardiovascular disorders such as atherosclerosis, coronary artery disease, and peripheral vascular disease. Traumatic injury and chronic diseases such as diabetes can also damage vascular tissue required for the perfusion of distal structures. Typical techniques used to treat such conditions include balloon angioplasty to restore patency of atherosclerotic vessels and surgical grafting of patent autologous blood vessels to replace occluded or damaged segments of vessels. Although angioplasty is widely used to restore vascular flow to poorly perfused tissues, its therapeutic effects are often quickly undermined by the subsequent restenosis of the vessel. Surgical grafting of autologous tissue is limited by the availability of adequate donor tissue in patients who are often suffering from widespread atherosclerotic lesions. Moreover, invasive surgical procedures often have limited beneficial effects because the grafted vascular tissue is commonly occluded by a thrombus or progression of atherosclerotic disease in the grafted vessel.

Currently available vascular bypass heterografts display adequate performance in the peripheral vascular circulation, yet autologous vein, when available, remains the graft of choice. Although synthetic grafts are more readily available and easily implantable, it is widely believed that autologous veins display enhanced patency over the long term. Indeed, biological grafts in general display a high freedom from thrombotic failure, yet often fail by pathologic remodeling leading to aneurysm formation or extensive intimal hyperplasia (an arterialization response in vein grafts). Memon et al., Cardiol. Rev.; 11(1):26-34, 2003; Baklanov et al., Vasc. Med.; 8(3):163-7, 2003. The therapeutic potential of biological grafts has driven an extensive research effort directed towards the correction of these aberrant remodeling responses.

The ideal vascular graft material would be one that is mechanically strong, suturable, biocompatible, and non-thrombogenic with the ability to remodel within the host. Mechanical strength of a heterograft is desirable so that it can be sutured and avoid rupturing after surgical implantation. Heterografts should also be biocompatible, in that they do not elicit an immune response after implantation or release toxic materials into the circulation, and are non-thrombogenic. The heterograft may be porous to allow for tissue ingrowth after implantation.

A variety of attempts have been made to develop a heterograft suitable for use as a replacement for blood vessels. However, these heterografts have had limited success.

Synthetic heterografts, for example those constructed from polytetrafluoroethylene (PTFE), such as Goretex or Teflon, or synthetic polyesters, such as Dacron, have been used primarily for replacing larger diameter blood vessels. However, synthetic grafts typically do not function well as replacements for smaller diameter vessels. Small diameter synthetic grafts typically suffer from problems such as having low infection resistance, inducing thrombosis, and providing insufficient burst strength, compliance, porosity, elasticity, and radial strength.

Thrombosis and long-term biocompatibility remain significant limitations to currently available vascular graft materials. The design of biologically based scaffolds, capable of supporting the growth of vascular cells and ultimately integrating with the host tissue, represents an attractive alternative. Recently a number of scaffolds have been developed for use in tissue engineered blood vessels, including collagen-based gels which lack sufficient mechanical strength, and collagen-rich matrices obtained from animal sources, which have limitations due to low endothelialization rates and high thrombogenicity. Furukawa et al., Cell Transplan; 11(5):475-80, 2002; He et al, Tissue Eng.; 8(2):213-24, 2002; He et al., Cell Transplant; 11(1):75-87, 2002; Kanda et al., Asaio J.; 39(3):M561-5, 1993; Tranquillo, Ann. N.Y. Acad. Sci.; 961:251-4, 2002; Skalak et al., Ann. N.Y. Acad. Sci.; 961:255-7, 1001; Badylak et al., J. Surg. Res.; 47(1):74-80, 1989; Roeder et al., J. Biomed. Mater. Res.; 47(1):65-70, 1999; Roeder et al., Biomed. Instrum. Technol.; 35(2): 110-20, 2001; Sandusky et al., Am. J. Pathol.; 140(2):317-24, 1992; Woods et al., Biomaterials; 25(3):515-25, 2004.

A number of studies have been performed using the submucosa of vertebrates as a material for preparing vascular grafts. Small intestinal submucosa (SIS) is a collagen based extracellular matrix scaffold and well established biomaterial that is currently commercially available (Cook Biotech Inc., West Lafayette, Ind.) for applications such as hernia repair, urethral treatment and wound care. Badylak et al., J. Surg. Res., 47(1):74-80, 1989.

The mechanical properties of SIS are well-matched to vascular applications, given that SIS bilayer tubular constructs have burst pressures that are comparable to those of native arteries. The compliance of typical SIS heterografts is just below that of native carotid arteries and an order of magnitude greater than synthetic heterografts. Roeder et al., J. Biomed. Mater. Res.; 47(1):65-70, 1999. SIS alone as a vascular graft has shown some promising results but requires rigorous anticoagulation therapy to prevent thrombosis and to establish an endothelial cell layer. Sandusky et al., Am. J. Pathol.; 140(2):317-24, 1992; Huynh et al., Nat. Biotechnol.; 17(11):1083-6, 1999; Sandusky et al., J. Surg. Res.; 58(4):415-20, 1995. Moreover, SIS has been prone to the formation of aneurysms several months after implantation. Opitz et al., Cardiovasc. Res.; 63(4):719-30, 2004.

A number of efforts have been made to improve the performance of heterografts, including seeding the grafts with endothelial cells (ECs) to provide a more natural graft in hopes of reducing thrombosis. U.S. Published Application 2003/0216811. Recent studies suggest that molecular approaches may indeed prove to be beneficial in limiting vein graft hyperplasia. Mann et al., Proc. Natl. Acad. Sci. USA; 92(10):4502-641,42, 1995; Ehsan et al., Circulation; 105(14):1686-92, 2002. However, such cellular modifications will not correct a key defect in current biological grafts—the absence of an appropriate arterial extracellular matrix.

In natural vasculature, fibroblasts secrete elastic biomolecules such as elastin, a 67 kDa extracellular matrix protein. Elastin is a major structural component of elastic arteries and is organized into a complex three dimensional structure principally consisting of concentric layers of interconnected fenestrated fibrous sheets, the hallmark of the distinct arterial lamellar structure. Clar et al., Arteriosclerosis; 5(1):19-34, 1985; Wolinsky et al., Circ. Res.; 20(1):99-111, 1967. Mechanically, elastin is a principal tissue component responsible for energy storage and recovery, and contributes to the unique dynamic tensile mechanical properties of arteries. Roach et al., Can. J. Biochem. Physiol.; 35(8):681-90, 1957.

Pathologic loss of elastin has been associated with end stage aneurysm disease in older adults and deficiency in elastin expression associated with supravalvular aortic stenosis in children. Baxter et al., J. Vasc. Surg.; 16(2): 192-200, 1992; Gandhi et al., Surgery; 115(5):617-20, 1994; Hunter et al., Proc. Soc. Exp. Biol. Med.; 196(3):273-9, 1991; Rizzo et al., J. Vasc. Surg.; 10(4):365-73, 1989; Curran et al., Cell; 73(1):159-68, 1993; Ewart et al., J. Clin. Invest.; 93(3):1071-7, 1994; Li et al., Hum. Mol. Genet.; 6(7):1021-8, 1997; Urban et al., Hum. Genet.; 104(2):135-42, 1999. Experiments in knock out and haploinsufficient mice have demonstrated elastin to be a very potent regulator of smooth muscle cell (SMC) phenotype and blood pressure. Broder et al., Am. J. Med. Genet.; 83(5):356-60, 1999; Eronen et al., J. Med. Genet.; 39(8):554-8, 2002; Faury et al., J. Clin. Invest.; 112(9):1419-28, 2003; Karnik et al., Development; 130(2):411-23, 2003; Li et al., Nature; 393(6682):276-80, 1998; Li et al., J. Clin. Invest., 102(10):1783-7, 1998; Rose et al., Eur. J. Pediatr.; 160(11):655-8, 2001.

As a biomaterial, elastin has several favorable properties, but use of pure elastin conduits has been limited by its low ultimate tensile strength and the difficulty of reconstituting an appropriate fiber structure. For example, Gregory et al., in Lasers in Surgery and Medicine; 35:201-205, 2004, disclose an elastin based matrix material derived from an arterial source for use as a heterograft. While this material was reported to be successfully grafted into swine, several heterografts burst after implantation, apparently lacking sufficient mechanical strength for long term use.

In several references, including U.S. Pat. No. 6,110,212, Gregory et al. describe the formation of biomaterials, for use as patches, formed from molded solutions of intermixed collagen and elastin. Methods of digesting arteries to prepare collagen-free biomaterials are also discussed. Kelly et al., in U.S. Published Application 2003/0118560, discuss multilayer grafts having collagen, proteoglycan and elastin layers, and teach that layers of pure elastin have insufficient structural integrity, and should be mixed with collagen to provide suitable strength to the layer.

In U.S. Pat. No. 6,667,051, Gregory discusses a patch having a layer of elastin sandwiched between layers of collagen. This material does not appear to be discussed as a potential vascular graft.

Berglund et al., in U.S. Published Application 2003/0072741, discuss an elastin scaffold formed into a cylinder and placed in a collagen gel such that collagen is adhered to the inner and outer surfaces of the elastin cylinder. However, it has been observed that such collagen gels are not as mechanically strong as collagen matrixes present in, or isolated from, biological sources. Mitchell et al., Cardiovascular Pathology; 12:59-64, 2003.

### SUMMARY

Many of the foregoing problems have been addressed by the graft prosthesis disclosed herein.

In one aspect, a composite multi-layered graft prosthesis includes a lumen-forming inner layer of elastin, and a separate outer layer having a sufficient amount of collagen matrix adhered to the inner layer to provide mechanical strength to the inner layer when the prosthesis is implanted in the body. In particular examples, the inner layer of elastin consists essentially of acellular elastin, or substantially pure elastin. The elastin may be obtained from a natural source or synthetically manufactured. When obtained from a natural source, the elastin may be obtained from a blood vessel, for example by chemical treatment of a blood vessel having elastin in its wall. The outer collagen matrix is, for example, acellular collagen, such as acellular submucosa, for example, acellular small intestinal submucosa. The outer and inner layer may be adhered to one another by an adhesive, such as a bio-adhesive, for example fibrin, or may be crosslinked, such as by chemical crosslinking. Growth factors may optionally be added to the graft, for example by incorporating the growth factors into the adhesive, to promote ingrowth of target cells into the graft.

The graft is particularly suited for use as an artificial blood vessel. The substantially pure inner layer of acellular elastin provides a relatively non-thrombogenic surface that helps maintain patency of the graft after implantation, while the acellular layer of collagen that surrounds the elastin layer has been found to provide suitable structural integrity to the graft to overcome many prior problems with pure elastin grafts.

The graft can be shaped into a tubular structure of appropriate caliber for surgical anastomosis as a segment between cut ends of a blood vessel. The graft is therefore suitable for use in methods of surgical anastomosis in which a segment of vasculature is removed to provide an anastomotic end of a blood vessel. The composite graft is placed proximate the anastomotic end of the blood vessel and anastomosed in place, for example by suturing the ends of the blood vessel to the ends of the graft, establishing patent flow through the graft prosthesis and vasculature.

Methods of constructing a composite graft prosthesis are also disclosed in which a collagen matrix (such as acellular small intestinal submucosa) is isolated from the tissue of an animal and wrapped about an elastin matrix also isolated from an animal. The formed composite graft has an inner surface of substantially pure elastin that is substantially free of collagen. The elastin can be wrapped around a mandrel as it rotates to form a tubular vessel, and the collagen may in turn be wrapped around the elastin layer to form the composite, multi-layer graft. An adhesive, such as fibrin, can be applied on the elastin layer before the collagen layer is applied to help form an integral fused structure that provides superior structural strength to the graft.

### BRIEF DESCRIPTION OF THE DRAWINGS

FIG. 1 is a schematic fragmentary perspective view of a two layer composite graft described herein.

FIG. 2 is a view similar to FIG. 1 but illustrating a two layer composite graft having an intermediate adhesive layer.

FIG. 3 is a digital image of an elastin matrix obtained by treating porcine carotid arteries with ethanol and sodium hydroxide.

FIG. 4 is a digital image of a sheet of small intestinal submucosa obtained from porcine small intestine.

FIG. 5 is a digital image showing a cross sectional end view of a vascular graft made in accordance with the principles described herein.

FIG. 6 is an enlarged digital image of the composite vascular graft from FIG. 5 showing more detail of the inner elastin and outer small intestinal submucosa layers.

FIG. 7A is a photomicrograph of Movat's Pentachrome staining of the wall of a disclosed composite graft. The inner layer of elastin is stained black and outer layers of collagen are more lightly stained (stained yellow in the original). The fibrin glue (stained red in the original) is seen layered between the elastin layer and the first layer of collagen, as well as between the two collagen layers.

FIG. 7B is a photomicrograph from a polescope, a microscope that quantitatively measures birefringent collagen fibers, of a disclosed composite graft. The brighter fibers on the right are the collagen layers of the SIS attached to the elastin of the composite vascular graft conduit.

FIG. 8 is a photomicrograph of a Fibrin II staining of the wall of a disclosed composite vascular graft to illustrate the depth of penetration of the fibrin glue into the elastin matrix.

FIG. 9A is a scanning electron microscopy image of a disclosed composite vascular graft prior to implantation.

FIG. 9B is a scanning electron microscopy image of a disclosed composite vascular graft after implantation, demonstrating patency.

FIG. 10 is a graph showing a stress-strain curve of a disclosed composite vascular graft.

FIG. 11 is a graph showing the burst pressure of an elastin graft and several composite vascular graft formulations for purposes of comparison.

FIG. 12 is a graph showing the failure force needed to cause suture failure for various combinations of native arteries, an elastin graft, and a disclosed composite graft.

FIG. 13 is a digital image of a disclosed composite vascular graft implanted in a swine and illustrating that the diameter of the graft is substantially the same as the native artery.

FIG. 14 is a digital image of a disclosed elastin composite vascular scaffold as a carotid interposition graft after a six hour implantation.

FIG. 15 is a graph showing the changes in activated clotting time (ACT) during six elastin composite vascular scaffold swine implantations. The changes in ACT demonstrate the return of the swine to baseline after a heparin dose of 100 U/kg was given prior to the implantation of the composite vascular grafts.

FIG. 16 is a graph of occlusion times for implanted composite vascular grafts and ePTFE control grafts, illustrating increased occlusion times for disclosed composite vascular grafts.

FIG. 17A is a digital image of a disclosed patent composite graft after explantation.

FIG. 17B is a digital image of an occluded disclosed composite graft after explantation.

FIG. 17C is a digital image of an occluded ePTFE control graft after explantation.

FIGS. 18A-18C are photomicrographs of a histologically stained composite graft after implantation and explantation.

FIG. 18D is a photomicrograph of a histologically stained ePTFE control graft after implantation and explantation.

### DETAILED DESCRIPTION

I. Abbreviations
HEPES—(N-[2-Hydroxyethyl]piperazine-N′-[2-ethanesulfonic acid])
EC—endothelial cells
ACT—activated clotting time
SIS—small intestinal submucosa
aSIS—acellular small intestinal submucosa
PTFE—polytetrafluoroethylene
ePTFE—expanded polytetrafluoroethylene
PBS—phosphate buffered saline
NaCl—sodium chloride
NaOH—sodium hydroxide
UTS—ultimate tensile strength
   II. Terms

In order to facilitate an understanding of the embodiments presented, the following explanations are provided.

Unless otherwise explained, all technical and scientific terms used herein have the same meaning as commonly understood by one of ordinary skill in the art to which this disclosure belongs. In case of conflict, the present specification, including explanations of terms, will control. The singular terms “a,” “an,” and “the” include plural referents unless context clearly indicates otherwise. Similarly, the word “or” is intended to include “and” unless the context clearly indicates otherwise. The term “comprising” means “including;” hence, “comprising A or B” means including A or B, or including A and B. Although methods and materials similar or equivalent to those described herein can be used in the practice or testing of the present disclosure, suitable methods and materials are described herein. The disclosed materials, methods, and examples are illustrative only and not intended to be limiting.

An “acellular” structure is one that is substantially free of cells. Hence acellular elastin is a layer that is substantially free of cells, such as at least 95%, at least 98% or 100% free of cellular material.

“Compliance” refers to elastic yield when a force is applied, for example as measured by the ratio of the change in the diameter of a blood vessel, or replacement therefor, to the change in pressure of the vessel. For example, blood vessels typically expand and contract in response to pressure changes caused by the change in blood pressure during a cardiac cycle.

A “composite” structure is one made of distinct parts, such as an elastin layer and a collagen layer. The distinct parts do not need to be separated by a definite border, but can have approximate or indistinct boundaries.

A “lumen” is the cavity of a tubular organ or the bore of a tube (such as the blood carrying portion of a natural or artificial blood vessel). A lumen-forming surface is a surface that is or can form the walls of a lumen.

An “inner” layer is a layer closer to the lumen, and an “outer” layer is a layer that is positioned on or around the inner layer. There may be multiple inner or outer layers. An outer layer need not be the outermost layer.

“Matrix” refers to the extracellular structure of a tissue or a layer thereof, including the arrangement, composition, and forms of one or more matrix components, such as proteins, including structural proteins such as collagen and elastin, proteins such as fibronectin and laminins, and proteoglycans. The matrix may comprise fibrillic collagen, having a network of fibers.

“Biological source” refers to an organism, such as an animal, such as a mammal, from which biological materials may be obtained. Examples of such materials include tissue samples, cells, extracellular material, or other organic or inorganic material found in the organism.

“Tissue” refers to an aggregate of cells usually of a particular kind together with their intercellular substance that form one of the structural materials of an animal and that in animals include connective tissue, epithelium, muscle tissue, and nerve tissue.

“Subject” refers to an organism, such as an animal, on whom experiments are performed or to whom treatments are administered. Subjects include humans, pigs, rats, cows, mice, dogs, and primates.

“Reconstituted” refers to material that is obtained from a biological source and processed so that it has a different form than that naturally found in the biological source. For example, reconstituted collagen or elastin is collagen or elastin that has lost an original matrix structure it contained in its natural form.

The above term descriptions are provided solely to aid the reader, and should not be construed to have a scope less than that understood by a person of ordinary skill in the art or as limiting the scope of the appended claims.

III. Overview

In certain examples, disclosed grafts have a collagen layer proximate to an elastin layer. In one implementation, graft material is formed into a tubular composite graft having a luminal elastin layer and an outer collagen layer which provides mechanical strength to the composite graft. In other implementations, the graft has multiple collagen and/or elastin layers. For example, a tubular graft may have a luminal elastin layer surrounded by a plurality of outer collagen layers. In other aspects, a tubular graft may have multiple alternating layers of collagen and elastin.

An elastin-collagen graft is believed to be beneficial because it may mimic native blood vessels. Native blood vessels typically have an inner elastin layer to provide elastic recoil and establish a biocompatible blood-contacting surface. Native blood vessels typically have an outer collagen layer to provide the required mechanical tensile strength.

Collagen layers may be formed from any suitable collagen source. In one implementation, the collagen layer is a collagen matrix isolated from an organism, such as the submucosa of a vertebrate. For example, the small intestinal submucosa of a vertebrate, such as a pig, may be treated to yield a collagen matrix suitable for use in the disclosed grafts. However, other sources of submucosa may be used, including pericardium or tissue from the alimentary, urinary, respiratory, or genital tracts of an animal. In other embodiments, the collagen layers are structurally substantially similar to the collagen matrix found in native vascular tissue. Such layers may include woven collagen fabrics or layers formed from collagen gels or solutions. In more particular examples, the collagen is synthetic or reconstituted collagen. The collagen matrix may comprise fibrillic collagen. If collagen gels or solutions are used, they may be formed into appropriate shapes, such as by using a mold. However the currently preferred source of collagen is acellular mucosal collagen, such as intestinal mucosa, for example small intestinal mucosa. Jejunal submucosa is an example of a suitable material.

Elastin layers may be formed from any suitable natural or synthetic source. In certain disclosed examples, the elastin is substantially pure, or in any event is substantially free of collagen. In one implementation, the elastin layer is an elastin matrix isolated from an organism, such as from the vascular tissue of a vertebrate. For example, the arterial tissue of a vertebrate, such as a pig, may be treated to yield an elastin matrix suitable for used in the disclosed grafts. In other embodiments, the elastin layer is structurally substantially similar to the elastin matrix found in native vascular tissue. The layer may be a woven elastin fabric or formed from an elastin solution or gel. In more particular examples, the elastic is synthetic or reconstituted elastin. The elastin matrix may comprise fibrillic elastin. If elastin solutions or gels are used, they may be formed into appropriate shapes, such as by using a mold.

An adhesive may be used to secure an elastin layer to a collagen layer. In one implementation, the adhesive is a fibrin glue, such as formed by the action of thrombin on fibrinogen. In one example, the collagen material and the elastin material are soaked in a fibrinogen solution and thrombin is added while the collagen layer is placed on the elastin layer. Other adhesives may be used rather than fibrin glue.

In at least certain aspects, the adhesive may be omitted and the collagen layer secured to the elastin layer by other means, such as staples, sutures, clips, or by a pressure, or friction, fit between the collagen and elastin layers.

The collagen and elastin layers may also be crosslinked together. One or both of the collagen and elastin layers may be internally crosslinked. Crosslinking may be used to alter the physical, structural, or mechanical properties of the graft, such as its compliance, burst pressure, or porosity. Crosslinking may be accomplished photolytically, chemically, by dehydration induced protein crosslinking, thermally, by radiation, or by other methods. If chemical crosslinking is used, any suitable crosslinking agent may be used, such as, for example, glutaraldehydes, genipen, denacols, Factor XIII, carbodiimides, ribose or other sugars, acyl-azide, sulfo-N-hydroxysuccinamide, or polyepoxide compounds.

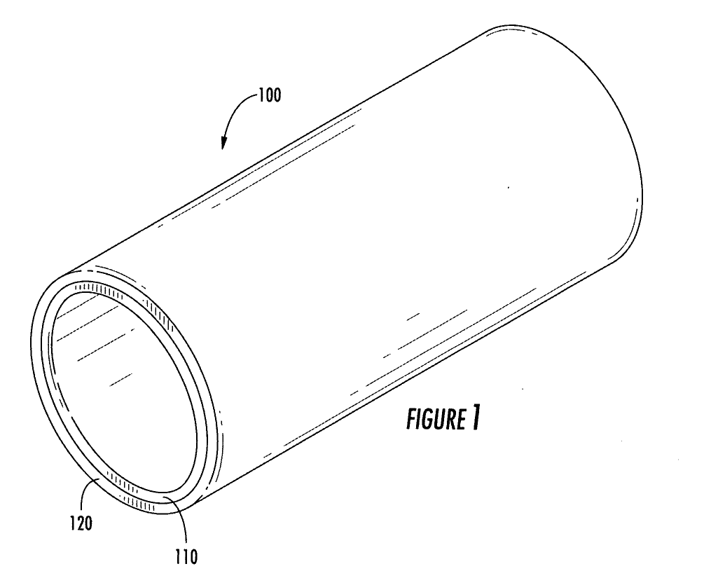

In one particular example, as shown in FIG. 1, a tubular graft 100 contains a luminal elastin layer 110 and an outer collagen layer 120. In one implementation, the elastin layer 110 is a matrix derived from a first biological source and the collagen layer 120 is a separate matrix derived from a second biological source. In another implementation, the elastin layer 110 is synthetic or reconstituted elastin derived from a first biological source and the collagen layer 120 is a matrix derived from a second biological source.

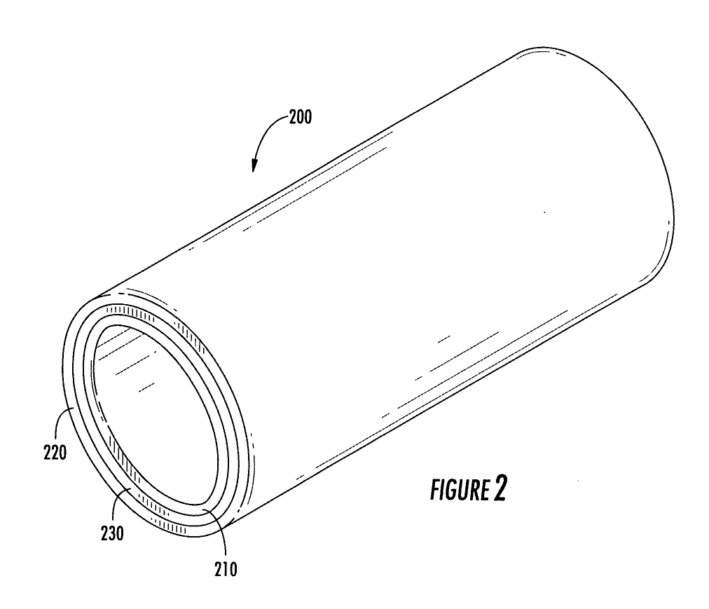

In another embodiment, as shown in FIG. 2, a tubular graft 200 contains a luminal elastin layer 210 and an outer collagen layer 220. An intermediate adhesive layer 230 is provided between the elastin layer 210 and the collagen layer 220 to secure the elastin layer 210 to the collagen layer 220. In one implementation, the elastin layer 210 is derived from a first biological source and the collagen layer 220 is derived from a second biological source. The elastin layer 210 and/or the collagen layer 220 may be a matrix. In another example, the elastin layer 210 is synthetic or reconstituted elastin and the collagen layer 220 is derived from a biological source and may be a matrix. In another example, the collagen layer 220 is synthetic or reconstituted collagen and the elastin layer 210 is derived from a biological source and may be a matrix. Although FIGS. 1 and 2 show single elastin and collagen layers, multiple layers of either or each could be used.

Disclosed grafts may be formed with physical properties tailored for a specific application. Preferably, the graft properties are chosen to correspond to native tissue which they will replace or augment. For example, the thickness of the graft, or particular layers of the graft, may be chosen to provide similar mechanical properties, including strength (such as measured by burst pressure, when the graft is a tubular graft), compliance, elasticity, and porosity, as native material. The size of the graft, including the diameter and thickness of a tubular graft, may be chosen to match the native tissue to which the graft will be connected.

Although any suitable layer or graft thickness may be used, typical graft thicknesses are about 25 microns to about 10 millimeters. For example, the grafts (or layers thereof) often have thicknesses of about 200 microns to about 5 millimeters, for example about 100 microns to about 1 millimeter. The thickness of each layer of the graft may be the same or different. The relative thickness of elastin and collagen layers may be varied to provide differing characteristics to the graft, such as elasticity and strength. In particular disclosed examples, the elastin layer is substantially pure elastin and is free of collagen.

Vascular grafts are preferably suitably strong and able to withstand the blood pressure of their environment after their implantation. For example, certain disclosed vascular grafts have a burst strength of at least about 500 mm Hg, for example 500 to about 2500 mm Hg, more preferably at least about 1000 mm Hg.

It is sometimes desirable to use graft materials that are sufficiently porous to allow in vivo remodeling or angiogenesis to occur, yet are not so porous as to allow undesired fluid leakage. One measure of porosity is the porosity index, which may be defined as the number of milliliters of water passed per cm²m⁻¹ at a pressure head of 120 mm Hg. In certain implementations, graft materials preferably have a porosity index of about 5 to about 50, more preferably at least about 10. For example, SIS materials typically have a porosity index of about 10 and woven Dacron typically has a porosity index of about 50. Pore sizes typically range from 2 to 500 microns, more typically 2 to 100 microns. The porosity of each layer may be the same or different.

Grafts are preferably sufficiently pure that they may be safely implanted in a subject, such as being sufficiently free of undesired pyrogens, endotoxins, microorganisms, irritative agents, hemolytic agents, carcinogenic agents, and infective agents. For example, the collagen and elastin layers preferably have an endotoxin level of less than about 12 endotoxin units per gram, more preferably less than about 1 endotoxin unit per gram. In at least one embodiment, the collagen and/or elastin layers are substantially acellular, such as having a nucleic acid content of less than about 2 micrograms per milligram. The layers preferably have a processing agent level of less than about 100,000 parts per kilogram. Suitable methods of measuring graft material purity, and of preparing a suitably pure collagen matrix layer, are discussed in U.S. Pat. No. 6,206,931.

When the graft includes a collagen matrix layer, the collagen matrix may be obtained from the submucosal tissue of a vertebrate. The procedures for obtaining suitable submuscally derived collagen matrices have been previously described. For example, U.S. Pat. No. 4,956,178 describes the preparation of a collagen matrix comprising the tunica submucosa, the lamina muscularis mucosa, and the stratum compactum (collectively referred to as the small intestine submucosa, or SIS) layers of the small intestinal tissue of warm-blooded vertebrates, such as pigs and cows. Briefly, small intestine tissue was subjected to a series of abrading steps to remove undesired portions of the small intestine. After a saline rinse and a brief (20 minute) soak in an antibiotic solution, such as 10% neomycin sulfate, the SIS material is ready for use. Other tissue can be used as the submucosa source, such as tissue from the stomach or urinary tract, such as discussed in WO 03/092381 and U.S. Pat. No. 6,485,723.

Alternatively, U.S. Pat. No. 6,206,931 discloses the preparation of a collagen matrix comprising primarily the tela submucosa from various animal sources, including from pig intestines. The source material, e.g. pig intestines, is first rinsed with a solvent, typically water. The material is then treated with a disinfecting agent, which is typically also an oxidizing agent. Peracetic acid is commonly used, although other agents can be used if desired, for example, hydrogen peroxide, chlorhexidine, or perpropionic acid. The disinfecting agent is typically used as an alcohol solution. The tela submucosa layer can then be delaminated from the tissue source and used.

An alternative collagen matrix layer to SIS, or similar tissues, is disclosed in U.S. Pat. No. 6,572,650. A single, aceullar layer of collagen may be obtained from various animals tissues, such as the tunica submucosa of the small intestine. The tunica submucosa is first separated from the source, such as by mechanically manipulating the material. The tunica submucosa is then cleaned, such as by treatment with a chelating agent, ethylenediaminetetraacetic acid tetrasodium salt, for example, under basic conditions. The material is then treated with an acid and a salt, such as hydrochloric acid and sodium chloride. The material is then treated with a buffered salt solution, such as a phosphate buffered saline solution, and rinsed with water. The collagen material obtained by this method typically contains very little substances other than collagen and a substantially intact collagen matrix.

The collagen material to be incorporated into the graft may be sterilized prior to its incorporation by any conventional method, including those disclosed in U.S. Pat. No. 6,572,650. For example, the material may be tanned using glutaraldehyde or formaldehyde. The material may be treated with ethylene oxide, propylene oxide, gamma radiation, gas plasma, or an electron beam. Alternatively, the collagen material can be treated with a basic solution of peracetic acid followed by rinsing with water. More than one sterilization technique may be used.

In certain aspects, the collagen material does not contain a natural matrix, such as synthetic collagen, gels and solutions of collagen, reconstituted collagen, and collagen fabrics. For example, gels or solutions of collagen may be used to form a collagen layer. For example, collagen materials, such as SIS, may be digested, for example with a protease. In another example, the collagen material (such as SIS) may be comminuted, such as by freeze drying the material and then grinding it into a powder. Examples of such procedures are discussed in U.S. Pat. No. 6,206,931. Another collagen source is acid digested rat-tail collagen, as described in U.S. Published Application 2003/0072741.

Collagen gels or solutions can be formed into layers, for example by treatment with a weak base to initiate fibrillogensis. Objects can be coated with collagen by inserting the object into a container of the collagen/base mixture. Exemplary methods of forming collagen coated materials are discussed in U.S. Published Application 2003/0072741.

The disclosed graft materials also include a layer of elastin. When an elastin matrix layer is to be used, it may be obtained from any suitable tissue containing an elastin matrix. Various sources of elastin matrix containing tissue, and methods for its isolation from surrounding tissue, are discussed in U.S. Pat. No. 5,990,379 and U.S. Published Application 2003/007241. For example, an elastin matrix may be isolated from arterial tissue, such as from a pig, by soaking the tissue in a saline solution (0.9% NaCl) overnight followed by sonicating the tissue for about two hours in a basic solution (such as 0.5 M sodium hydroxide).

In other implementations, elastin solutions or gels, woven elastin, or reconstituted elastin may be used to form an elastin layer. U.S. Published Application 2003/007241 and U.S. Pat. No. 5,990,379, and references cited therein, discuss materials and methods for forming elastin layers from various natural and synthetic sources. If desired, additional components, such as fibrin, collagen, cellulose derivatives, and calcium alginate, may be added to increase the mechanical strength of the elastin layer. Similarly, adhesive proteins may be added to increase mechanical, adhesive, or elastic properties of the elastin material. For example, proteins such as the von Willebrand factor, thrombospondin, laminin, or the FVIII complex may be added to the elastin layer, as discussed in U.S. Pat. No. 5,223,420.

Molds may be used to form collagen or elastin solutions or gels into a desired form, including sheets and tubes. The molds can be used to form the gels into a desired thickness, typically between 10 microns and 10 millimeters. The thickness of the layers may be varied according to the tissue it will replace. Preferably, the layers are of a similar thickness as corresponding layers occurring in the native tissue and the overall graft has a similar thickness to the tissue the graft will replace.

For certain grafts, an initial composite sheet is prepared having at least a collagen layer and an elastin layer, and optionally an adhesive layer securing the collagen layer to the elastin layer. The composite sheet can be formed into a cylindrical shape for use as a vascular graft by wrapping the composite sheet around a mandrel.

In other examples, an elastin sheet is first wrapped around a mandrel one or more times to form an elastin layer and then a collagen sheet is wrapped one or more times over the elastin sheet. Additional wrappings of collagen or elastin can be made, if desired. In one implementation, a graft is formed by wrapping an elastin matrix layer around a mandrel, followed by wrapping a sufficient amount of a collagen matrix sheet around the elastin layer to form two collagen layers.

In one example the mandrel is a sterile glass rod. The mandrel may be made from any other suitable medical grade material, including stainless steel or Teflon. The mandrel is preferably curved, such as having a circular or elliptical cross section, and typically has approximately the same diameter as a blood vessel that is to be replaced.

After wrapping the composite sheet, or elastin or collagen layers, around the mandrel, excess material may be removed and the sides of the material may be secured together, such as by suturing, stapling, clips, or other means.

In another implementation, material is wrapped around the mandrel so that there is a small overlap, such as between about 5% and about 20%, of material. The overlapped portions of the material may be secured together, such as by dehydration, including under atmospheric or vacuum conditions, or by chemical means, for example as discussed in U.S. Pat. No. 5,997,575. Pressure may be applied to the bonding region during the dehydration process.

The sheet of composite material, or a sheet of collagen or elastin, may be wrapped around the mandrel multiple times to create a thicker graft wall or a thicker collagen or elastin layer. The thickness of the graft wall, or the layer, may be selected to match the tissue which the graft will replace. For example, the thickness of each layer and the overall graft thickness may be selected to provide a graft having similar strength, porosity, elasticity, and compliance to the native tissue. Preferably, an additional, about 5% to about 20%, of material overlap is used to serve as a bonding region. The angle of wrapping and overlap, if any, between wrappings may be varied as desired in order to form a graft having particular structural, mechanical, and/or physical characteristics. Similarly, the size and shape of the material may affect the number of wrappings needed to form a complete layer of material over the mandrel (or other material covering the mandrel), and the properties imparted to the graft.

The mandrel may be covered with a material that aids in removal of the graft from the mandrel after formation. The material is preferably nonreactive towards any of the components of the graft and is a medical grade material that will not compromise the biocompatibility of the graft, such as medical grade synthetic materials including elastic, latex, Teflon, or rubber. After the graft is formed, the mandrel coating, and the graft along with it, may be slid off the mandrel.

In certain cases, the mandrel may be capable of expanding and contracting, such as a balloon, in order to aid in removing the composite graft from the mandrel without damaging the composite graft. In a further implementation, a mandrel may be omitted and barbs, or other securing devices, may be used to hold an elastin layer, such as a preformed tubular elastin layer, under tension. Additional layers may then be placed over the elastin layer. In a further example, material, such as fluid or gel, may be placed inside of a tubular elastin layer in order to help the elastin layer maintain its shape or position while additional layers are placed over the elastin layer.

The graft may also be constructed by other means. For example, tubes of elastin and collagen may be prepared. The elastin tube may then be pulled inside of the collagen tube to form a composite graft. The elastin tube may be secured to the collagen tube. For example, the diameter of the elastin tube may be only slightly smaller than the collagen tube, resulting in a friction fit between the two tubes.

Regardless of the method of forming the composite graft from the collagen and elastin layers, the collagen layer may be secured to the elastin layer. In one example, the collagen and elastin layers are secured by physical means, such as sutures, clips, staples, and the like.

Chemical means may be used to secure the collagen and elastin layers, including crosslinking the layers and/or using an adhesive. Chemical means may also be used to secure multiple composite layers or windings to one another. When adhesives are to be used, the adhesive, or a component thereof, may be applied to one or more surfaces of the layers to be joined. Any adhesive having suitable binding and biocompatibility properties may be used, such as fibrin glue, proteinaceous adhesives, cyanoacrylate cement, gelatin or collagenous pastes, polyurethane, polyepoxy, vinyl acetate, or other medical grade adhesives. The adhesive may be applied by any suitable method, including by soaking the layers in an adhesive or by brushing, spraying or applying the adhesive with a syringe on the appropriate surfaces.

In particular, fibrin is a naturally occurring polymer and as such is non-toxic, biocompatible and resorbable. Fibrin sealants have been successfully used in surgical applications. The structure of the fibrin gel, its strength, rate and extent of polymerization can be regulated by temperature and the concentration of fibrinogen, thrombin, Factor XIII, or calcium. Galanakis et al., Biochemistry; 26(8):2389-400, 1987; Hardy et al., Ann. N.Y. Acad. Sci.; 408:279-87, 1983; Okada et al., Ann. N.Y. Acad. Sci.; 408:233-53, 1983.

There is generally a correlation between adhesive shear strength and fibrinogen concentration. Sierra et al., J. Biomed. Mater. Res.; 59(1):1-119, 2002; Marx et al., J. Lab. Clin. Med.; 140(3):152-60, 2002; Glidden et al., Clin. Appl. Thromb. Hemost.; 6(4):226-33, 2000. Commercially available fibrin sealants typically have a range of fibrinogen concentrations of 50-100 mg/ml and thrombin concentrations of 216-1247 U/ml. Dickneite et al., Thromb. Res.; 112(1-2):73-82, 2003. In one implementation, a fibrin formulation is used which has similar fibrinogen levels (e.g. 56 mg/ml) as commercial availably fibrin sealants, but is polymerized with lower thrombin concentrations (e.g., 10 U/ml).

Temperature may be used to regulate the speed of polymerization. By using low thrombin levels and applying the adhesive at room temperature, the polymerization speed may be slowed and the degree of adhesive penetration into the graft layers may be increased. Unlike in vivo situations where rapid hemostasis may be required, polymerization over a more prolonged curing period may increase the ease of manufacturing the grafts and their resulting burst pressure strengths. Although the fibrin adhesives produce grafts having adequate initial strength, the integrity of the scaffold over the long term also depends on cellular repopulation and consequent remodeling of the collagen matrix—both of which may be enhanced by the resorbable nature of the fibrin bond.

In one implementation, the layers to be incorporated into a graft are soaked in a fibrinogen solution. Thrombin is applied to the surface of the layers as the layers are placed into contact with one another. Alternatively, fibrinogen may be applied to the bonding surface of one of the layers and thrombin may be applied to the bonding surface of another layer and then both of the bonding surfaces are placed into contact and allowed to cure. The fibrin glue is preferably allowed to cure, such as between about two minutes and about twelve hours, so that it may reach its full adhesive strength. If desired, the graft may be treated with an anti-thrombin agent, such as PPACK (Calbiochem, San Diego, Calif.), prior to implantation to neutralize any remaining thrombin.

If the layers of the graft, including the collagen and elastin layers or multiple layers or windings of a composite material, are to be crosslinked, any suitable method may be used. Examples of crosslinking methods that may be used include photolytic crosslinking, chemical crosslinking, dehydration induced protein crosslinking, radiation treatment, or other methods. If chemical crosslinking is used, any suitable crosslinking agent may be used, such as, for example, glutaraldehydes, genipen, denacols, Factor XIII, carbodiimides, ribose or other sugars, acyl-azide, sulfo-N-hydroxysuccinamide, or polyepoxide compounds. One particularly useful chemical crosslinking agent is 1-ethyl-3-(3-dimethylaminopropyl)carbodiimide hydrochloride. Materials and methods of crosslinking collagen layers are discussed in U.S. Pat. No. 6,572,650.

Growth factors may be incorporated into certain disclosed grafts. Growth factors may be selected to encourage cellular ingrowth (or cellular remodeling) after the graft is implanted in a subject. Examples of growth factors that may be incorporated into the graft include basic fibroblast growth factor, epidermal growth factor, platelet derived growth factor, transforming growth factor—alpha and transforming growth factor—beta. In one implementation, the growth factors are incorporated into an adhesive used to secure the grafts layers to one another. For example, the growth factors may be added to a fibrinogen solution when the adhesive will be a fibrin glue. These growth factors may be crosslinked into the adhesive layer, if desired.

Other materials may be added to a graft to alter its structural or biological properties, including the graft's biocompatibility or ability to be bioremodeled in vivo. These materials may be added to the layers of the graft prior or subsequent to the formation of the graft. In one implementation, the graft layers may be coated with a material to reduce the incidence of thrombosis. For example, one or more of the graft surfaces may be coated with heparin, such as is described in WO 2004/022107 and U.S. Pat. No. 6,572,650. For example, the graft, or layers that will be incorporated into the graft, may be contacted with an isopropyl alcohol solution of benzalkonium heparin to ionically bond heparin to the graft layers.

Fibrin, fibrin degradation products, or other substances may be applied to what will be the surfaces of the graft in order to reduce thrombosis. The use of fibrin degradation products to reduce thrombosis is described in U.S. Pat. No. 5,693,098.

It is known that the natural endothelial cell lining of blood vessels helps to prevent thrombosis, reduce susceptibility to infection, and increase the duration of graft patency. It may be difficult to completely cover a graft with a layer of endothelial cells (ECs) prior to implantation, particular if autologous cells are to be used. Accordingly, the disclosed grafts may be seeded with ECs prior to implantation, if desired. Seeding the graft with ECs may result in the more rapid formation of a graft endothelial layer after the graft is implanted in a subject.

ECs may be obtained from any suitable source, such as saphenous vein or umbilical vein. Exemplary methods of obtaining ECs and seeding them into grafts are discussed in U.S. Pat. No. 5,693,098; U.S. Pat. No. 6,503,273; U.S. Pat. No. 5,131,907; U.S. Published Application 2003/0216811; and references cited therein. For example, ECs can be isolated from tissue, such as vascular or skin tissue. Preferably, autologous ECs are used to eliminate disease, rejection, or adverse reaction to the graft after its implantation. The ECs may be introduced into the graft by any suitable method, such as by cannula.

Elastin based graft materials are known to undergo calcification after implantation. This calcification can compromise a graft's usefulness. Accordingly, the elastin layer may be treated with an aliphatic alcohol, such as ethanol, prior to implantation, as discussed in U.S. Pat. No. 6,372,228.

The disclosed grafts may be implanted by any suitable method. When used as vascular grafts, the disclosed grafts may be implanted by any suitable method. In one implementation, the disclosed grafts are implanted during a vascular anastomosis procedure. In a typical end-to-end vascular anastomosis procedure, blood flow to the vascular tissue to be replaced is interrupted. The vessel is then surgically excised and removed from the subject. The vascular graft may then be anastomosed to the native tissue. For example, the proximal and distal ends of a disclosed vascular graft may be sutured to the native vascular tissue to establish a surgical margin that does not leak. Other means may be used to attach the vascular graft to native tissue, such as laser techniques, sleeves, coupling rings, and the like, including those described in U.S. Pat. Nos. 6,673,085 and 4,470,415, and references cited therein. After the vascular graft is in place, blood flow may be restored through the vascular graft. The vascular graft may also be used in an end-to-side anastomosis.

The disclosed grafts may find use in areas in addition to their use as vascular grafts. For example, flat sheets of the material may be used as grafts, including tissue grafts, skin grafts, stomach grafts, intestinal grafts, bladder grafts, organ grafts, and the like. Tubular grafts may be used in other contexts, such as grafts for the urinary tract, for repair or augmentation of tubular organs, as stents, and as coatings for other tubular prosthesis, such as metal or synthetic stents, fistulas, and the like.

The following examples are provided to illustrate certain particular features and/or embodiments, but these examples should not be construed to limit the invention to the particular features or embodiments described.

### EXAMPLE 1

### Formation of a Composite Vascular Graft

Preparation of Elastin and SIS

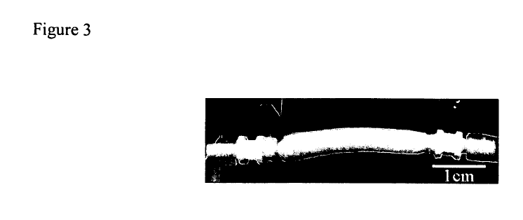

Porcine carotid arteries were obtained from domestic swine of approximately 250 lbs. (Animal Technologies, Tyler, Tex.). The arteries were shipped overnight in phosphate-buffered saline (PBS) on ice. The gross fat was dissected away and, using aseptic techniques, the arteries were placed in 80% ethanol for a minimum of 72 hours at 4° C. and subsequently treated with 0.25M NaOH for 70 min with sonication at 60° C., followed by two 30-minute, 4° C. washes in 0.05M HEPES (pH 7.4). The extracted elastin tubular conduits were then autoclaved at 121° C. for 15 minutes, and stored at 4° C. in 0.05M HEPES. An image of an elastin conduit is shown in FIG. 3.

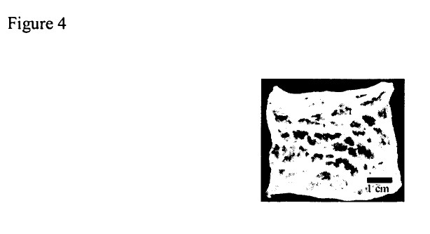

The submucosa was isolated by physical debridment of the small intestines of approximately 450 lbs domestic swine (Animal Technologies), as described by Badylak et al., J. Surg. Res.; 47(1):74-80, 1989. The SIS was then cut into two inch longitudinal segments, rinsed in 0.05M HEPES, treated for 90 minutes with 0.1M NaOH, rinsed in 0.05M HEPES, and stored in 10% neomycin sulfate. Prior to use, the tissue segments were rinsed with 0.05M HEPES, cut longitudinally, and opened to make a sheet, shown in FIG. 4. These acellular SIS sheets (aSIS) were then frozen to −80° C. and freeze-dried (FreeZone 6, Labconco, Kansas City, Mo.).

Graft Fabrication

Fibrin was used to bond the aSIS and elastin biomaterials. Initial experiments were performed to optimize fibrinogen concentration. Lyophilized bovine fibrinogen (Sigma, St. Louis, Mo.) was reconstituted with 0.1M Tris Buffer, pH 7.4 containing 0.09% NaCl to final concentrations of 30 and 56 mg/mL. The outer and inner surfaces of the elastin and aSIS biomaterials, respectively, were covered with the fibrinogen solution and incubated for 5 minutes at room temperature. Bovine thrombin (10 U/mL, Jones Pharma, Inc. St. Louis, Mo.) reconstituted in 0.1M Tris Buffer, pH 7.4 containing 0.09% NaCl and 5 mM CaCl₂ was added to a portion of the aSIS surface, which was then wrapped onto the elastin tubular conduit; additional thrombin was added to the aSIS surface as the wrapping progressed. The aSIS was wrapped twice around the elastin conduit with an additional 20% overlap. The elastin composite vascular scaffold was then placed in a 37° C., 75% humidity environment overnight. The composite scaffolds were then rehydrated in 0.05M HEPES.

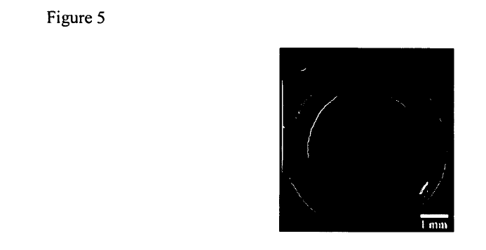

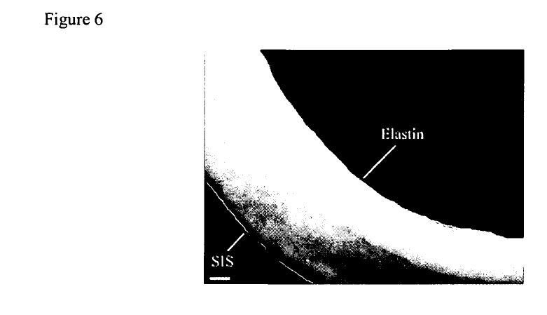

Examples of composite grafts, having average internal diameters of about 4 millimeters, are shown in FIGS. 5 and 6. In FIG. 6, the bar indicates 100 microns. Analysis of the Physical Properties of the Composite Graft

The structure of the elastin composite vascular scaffold was analyzed using histology and electron microscopy methods. Paraffin-embedded sections (5 μm thick) were stained with hematoxylin & eosin and Movat's Pentachrome to evaluate the consistency of the scaffold layers. Fibrin penetration into the elastin conduits was confirmed by immunostaining with a Fibrin II monoclonal primary antibody (Accurate Chemical & Scientific Corp., Westbury, N.Y.). The tissue was pretreated in a steamer for 20 minutes using 1 mM EDTA for antigen retrieval. The sections were run on an automated IHC stainer (Ventana Medical Systems, Inc., Tucson, Ariz.), incubated with the primary antibody at a dilution of 1:400 for 30 minutes, and then processed using the standard DAB kit (Ventana Medical Systems). Tissue samples for scanning electron microscopy (SEM) were fixed with 2.5% glutaraldehyde, freeze dried (Freezone 6, LabConco), sputter coated (Hummer II, Technics Inc., Alexandria, Va.), and viewed with a DSM 960 scanning electron microscope with a LaB₆ source (Zeiss, Oberkochen, Germany).

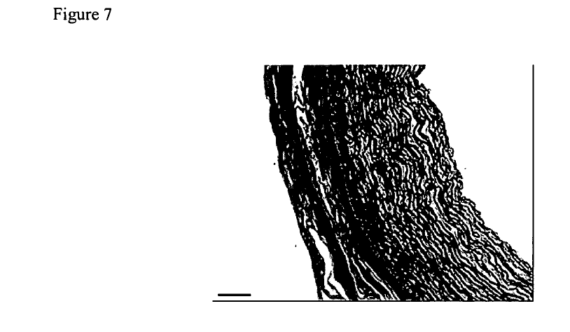

Over 200 composite vascular scaffolds have been constructed from porcine derived arterial elastin, fibrin glue, and sheets of aSIS. An example of such a scaffold is shown in FIG. 6. The composites displayed handling characteristics similar to native arteries. Histological examination, shown in FIG. 7A (Movat's Pentachrome stain, bar represents 100 microns), more clearly revealed the unique composite structure of the scaffold; the outer (adventitial) portion of the composite is composed of two layers of the predominantly collagenous aSIS (yellow) bonded together with a distinct band of fibrin (red) with a second band of fibrin bonding the aSIS to the purified lamellar elastin (black) structure comprising the media. FIG. 7B illustrates the fibrillic nature of the aSIS collagen layer.

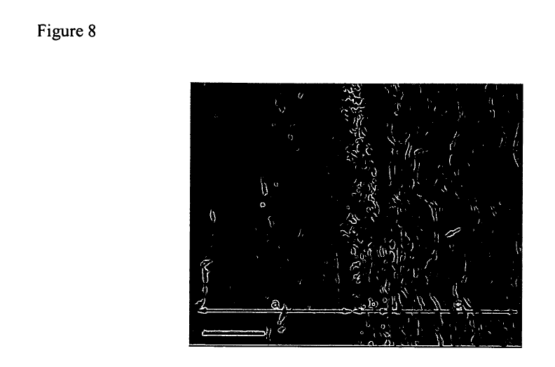

Bonding between layers is likely enhanced by a deep penetration of the fibrin into both the elastin and aSIS, as shown in FIG. 8, a Fibrin II staining (brown) of the elastin composite scaffold. Region (a) indicates the aSIS region, region (b) the transitional region, and region (c) has single arrows pointing to the elastin lamellae. The bar represents 10 micrometers.

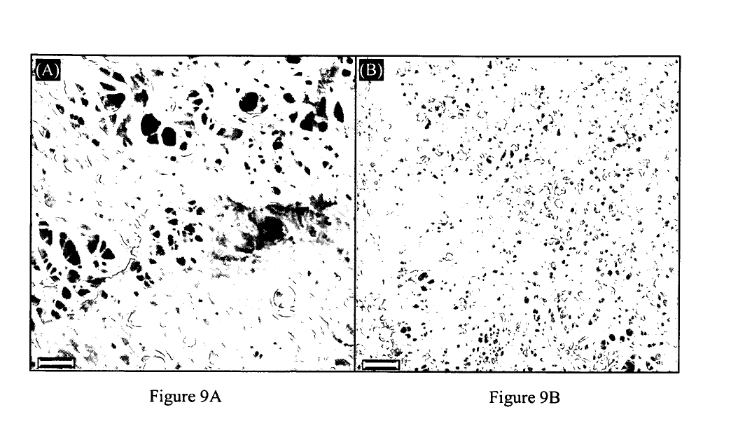

SEM of the internal composite scaffold surfaces, FIGS. 9A and 9B, show an intact elastin fibrillar structure typical of native porcine carotid arteries. FIG. 9A is a SEM image of the lumen of the elastin composite vascular scaffold indicating that the lamellar structure of native arteries is maintained in this matrix. The scale bar in FIG. 9A indicates 10 microns. The image was taken prior to implantation and indicates that the elastin fibers are 0.5 to 3 microns in diameter and the predominant axis of orientation is longitudinal.

FIG. 9B is a SEM image of the composite vascular scaffold after implantation and demonstrated patency. In the patent vessels, there was evidence of isolated platelet adhesion. The scale bar indicates 20 microns. The elastin fiber diameters were 0.5 to 3 microns with the predominant orientation in the longitudinal direction. In some regions, the fibrillar structure appears to fuse into a fenestrated sheet, in these regions fenestrations in the internal elastic lamellar unit range in size from 2 to 5 microns in these unstrained samples.

In Vitro Testing

Tensile Testing

Uniaxial tensile testing was performed on longitudinal sections of elastin tubular conduits and elastin composite vascular scaffolds, constructed with 30 mg/mL fibrinogen (n=6). Dog bone shaped samples were cut to a gauge length of 20-40 mm and width of 4-6 mm with the thicknesses of the samples between 0.40 and 0.55 mm. The test samples, hydrated with 0.05M HEPES, were preconditioned at 10±5% strain at a rate of 2 Hz and then ramped to failure at a rate of 5 mm/s (500 N load cell, Tytron Micromechanical Testing System, MTS, Inc., Eden Prairie, Minn.). Time, displacement, and force measurements from the MTS, as well as the sample dimensions, as measured by digital calipers, were input into a custom Matlab program to determine the engineering stress-strain curves. The ultimate tensile strength (UTS), maximum failure strain, and tangent modulus at 30% strain based on a fourth order polynomial fit were determined.

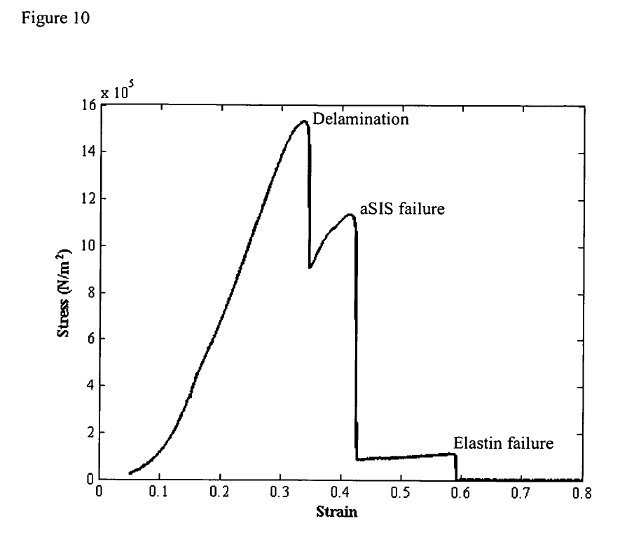

FIG. 10 illustrates a typical stress-strain curve of a dog bone shaped specimen of the elastin composite vascular scaffold, which was preconditioned and pulled to failure. As shown in FIG. 10, the stress-strain curve of the vascular scaffold contains profiles typical of a collagen and elastin composite material. The composite failed in three distinct phases with the initial failure point supporting the highest loading. We have interpreted the failure as the initial delamination of the aSIS layers, followed by breakage of individual aSIS layers, and finally low load failure of the elastin. This interpretation is supported by the observed mode of failure and the failure modes of the individual components, with the high collagen content in the aSIS supporting the highest loads and the final section typical of the more linear high strain failure of elastin. Sherebrin et al., Can. J. Physiol. Pharmacol.; 61(6):539-45, 1983. The results, summarized in Table I below, displayed an order of magnitude increase in both UTS (p<0.001, t-test) and tangent modulus (p<0.02, t-test) of the composite material over that of purified elastin.

Burst, Cyclic Circumferential Strain, and Peel Testing

Burst pressure testing was performed on both the elastin tubular conduits and the elastin composite vascular scaffolds (n=3 to 10). The ends of the scaffolds were fixed in position under zero longitudinal load and then saline was infused at a rate of 100 mL/min. The pressure and diameter were continuously monitored by an inline pressure transducer (Transpac IV Monitoring kit, Abbott Labs, N. Chicago, Ill.) and a video dimension analyzer (VDA 303, Vista Electronics, Ramona, Calif.) both input into a Macintosh Powerbook G4 running a data acquisition program developed with Labview software.

Burst pressure testing was performed on elastin tubular conduits, cut to 2.54 cm length with an average initial diameter of 5.53±0.54 mm, and three formulations of the elastin composite vascular scaffolds, including (A) 30 mg/mL fibrinogen with fully hydrated aSIS (3.64±0.75 cm length, 6.87±0.40 mm initial outer diameter), (B) 30 mg/mL fibrinogen with freeze dried aSIS (3.33±0.35 cm length, 7.06±1.29 mm initial outer diameter), and (C) 56 mg/mL fibrinogen with freeze dried aSIS (2.39±0.71 cm length, 6.50±0.27 mm initial outer diameter). Increasing the concentration of fibrinogen and lyophilizing the aSIS prior to attachment increased the burst strength.

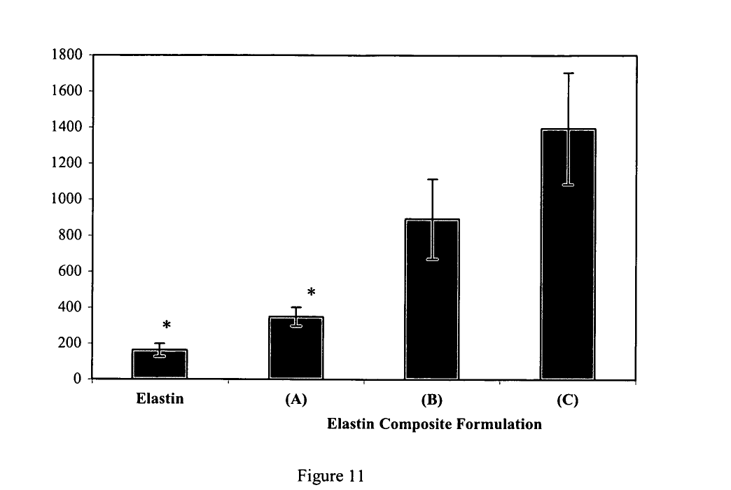

The conduits composed of elastin alone had an average burst pressure of 162±36 mmHg (n=10), while composite scaffold formulations had higher burst pressures of (A) 349±53 mmHg (n=3), (B) 894±222 mmHg (n=3), and (C) 1396±309 mmHg (n=9), summarized in Table I and FIG. 11. The burst pressure of formulation (C) was significantly higher than both the elastin alone and formulation (B) (p<0.001, ANOVA, Tamhane post-hoc). Thus, formulation (C) was strongest and was therefore used for all further testing. Elastin grafts had an average burst pressure very close to physiological arterial pressures, while the composite graft results were an order of magnitude higher.

Cyclic circumferential strain testing was performed using a vascular graft fatigue testing platform (9130-8 SGT, EnduraTEC, Minnetonka, Minn.). The composite vascular scaffolds were tested in a saline bath maintained at 37° C., internally pressurized with saline, and cycled at 1 Hz between 120 and 80 mmHg (n=5). The outer diameter was continuously recorded with a laser micrometer and the tests were run for a minimum of 300,000 cycles or 83 hours.

The cyclic circumferential strain test parameters were chosen to evaluate the scaffolds for gross delamination of the elastin and SIS layers under pulsatile conditions. All composite scaffolds held pressure, without leaks, for the entire test period of at least 83 hours.

Peel testing was performed by manufacturing the elastin composite vascular scaffold with the final portion of aSIS left as a free flap for gripping (n=8). Peel strength was determined by loading a 16 mm long scaffold on a mandrel and pulling the free flap at a 90° angle at a rate of 1 mm/s (Vitrodyne V1000, Chatillon, Greensboro, N.C.). The peel strength (N/mm) was calculated from the average force (N) divided by the specimen width (mm).

The peel testing determined that the average peel strength of the composite scaffold was 0.019±0.005 N/mm (n=8), as summarized in Table I, below.

TABLE I
Comparison of Mechanical Properties of Elastin Composite Vascular
Scaffold to Elastin Conduit

| | Elastin Composite | Elastin |
|---|---|---|
| Test | Vascular Scaffold | Conduit |
| Ultimate Tensile Strength (MPa) | 1.744 ± 0.278* | 0.196 ± 0.067 |
| Max Strain | 0.453 ± 0.172 | 0.571 ± 0.100 |
| Tangent Modulus at 30% Strain | 6.578 ± 3.181* | 0.268 ± 0.056 |
| Burst Pressure (mmHg) | 1396 ± 309* | 162 ± 36 |
| Peel Strength (N/mm) | 0.019 ± 0.005 | N/A |
| Cyclic (hours) | ≧83 | N/A |

*p < 0.02, compared to purified elastin conduit

Suture Pullout Strength

Composite vascular scaffolds, elastin tubular conduits, and native porcine carotid arteries were tested for their ability to retain sutures (n=5). Test samples were anastomosed in an end-to-end fashion using a running suture of 6-0 prolene mounted on a BV-1⅜ circle needle (Ethicon, Somerville, N.J.). The anastomosis was centrally located between the two pneumatic side action grips (Instron, Canton, Mass.) with a total specimen length between the grips of 22±3 mm. Tissue was maintained in a hydrated state at room temperature and tested to failure at a displacement rate of 5 mm/s (858 Mini Bionix II, MTS). Failure force was determined from the peak of the force-displacement curve. Each specimen was observed until failure, with the failure mechanism recorded.

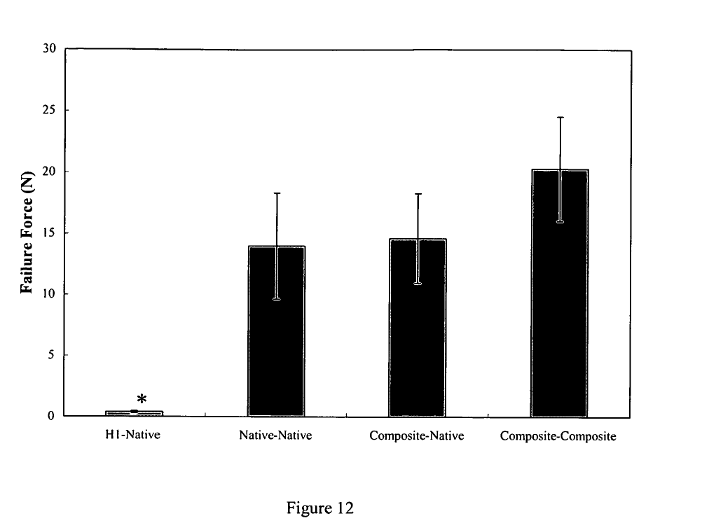

The addition of the fibrin-aSIS layers to the elastin biomaterial significantly increased the suture failure load. FIG. 12 illustrates the average suture failure forces of native arteries sutured to pure elastin conduits, native arteries, and elastin composite vascular scaffolds, as well as composite scaffolds sutured to composite scaffolds (average±standard deviation). When sutured to fresh porcine carotid arteries, pure elastin biomaterials failed at the suture line. In marked contrast, the reinforced elastin composite vascular scaffold did not fail, rather the native arteries were the point of failure. The composite vascular scaffold sutured to native arteries had an average suture failure load of 14.612±3.677 N, nearly 40 fold higher than that of the elastin biomaterial, 0.402±0.098 N (p<0.001, ANOVA, Bonferroni post-hoc). The suture failure load of the composite vascular scaffold-to-native artery samples was no different than the native-to-native artery, nor the composite-to-composite samples (ANOVA, NS).

### EXAMPLE 2

### Short Term In Vivo Testing

The composite grafts of Example 1, as carotid artery conduits, were implanted into 6 swine and observed for 20 minutes to evaluate the suturability and short-term thrombogenicity of the composite grafts. The composite graft has shown positive results by increasing the mechanical strength and suturability compared to that of a pure elastin graft.

The carotid artery was exposed, cut circumferentially, and a 1 cm segment was resected. The vascular graft was anastomosed to the carotid in an end to end fashion using 6-0 prolene running suture technique. After completing the anastomosis, proximal and distal clamps were released respectively. The graft was left in position for 20 minutes for the preliminary studies.

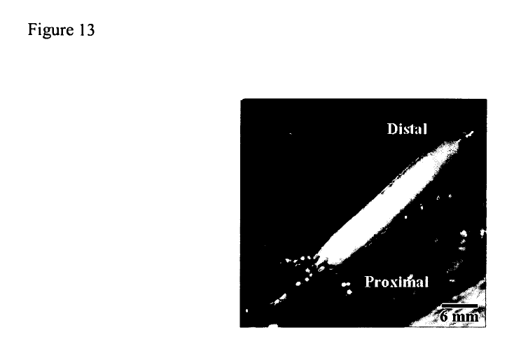

All composite grafts were sutured successfully in vivo and were 100% patent with no gross thrombi at excision. FIG. 13 is an image of an implanted composite graft. The diameter of the composite graft is closely matched to that of the native artery (indicated by arrows). The composite vascular graft was suturable and maintained patency under physiological conditions.

### EXAMPLE 3

### Acute Porcine Interposition Graft Study

Aseptic processing was used to manufacture six composite scaffolds for implantation into a swine model. In these scaffolds, the thrombin within the fibrin layers was inhibited by pretreatment with 0.15 ug of PPACK (Calbiochem, San Diego, Calif.) to block residual active thrombin. Spectrozyme TH Assays (American Diagnostica, Stamford, Conn.) confirmed that this concentration was sufficient for the amount of thrombin used (data not shown).

National Institutes of Health (NIH) guidelines for the care and use of laboratory animals (NIH Publication #85-23 Rev. 1985) were observed for all animal experiments. Bilateral carotid interposition grafts were implanted in a total of six domestic swine of approximately 220 lbs. Animals were sedated with Telazol 4-9 mg/kg IM and anesthesia was maintained with Isoflurane. Normal saline was delivered intravenously at a rate of 200 cc/hour. A femoral cutdown was performed and a 6-7Fr sheath placed to monitor blood pressure and to catheterize for angiography. O₂ saturation, blood pressure, temperature, activated clotting time (ACT), and heart rate were recorded every 30 minutes during the procedure.

Carotid arteries were exposed and treated with Papaverine:Lidocaine (1:4) solution to locally dilate the vessels. Intravenous heparin (100 units/kg) was given before cross-clamping the arteries and the ACT was maintained above 250 seconds during the graft implantation period. Doppler flow probes (Transonic Systems Inc., Ithaca, N.Y.) were placed distal to the anastomotic site and coupled to the artery using ultrasound jelly. The exposed carotid artery was cross-clamped, divided in the center, and 1 cm resected. The stumps of the artery were flushed with heparin, the grafts were implanted in an end-to-end fashion using a running suture of 6-0 prolene, and after completing the anastomoses, the proximal and distal clamps were released, respectively. This procedure was repeated for an ePTFE control graft (4 mm diameter, GORE-TEX®, W.L. Gore & Associates, Inc., Flagstaff, Ariz.) on the contralateral artery.

Angiography of both carotid arteries was performed after implantation and at study endpoint. At study endpoint, saline was infused into the carotids, the arteries were clamped and the grafts were explanted and rinsed.

Experimental Design

In each animal, the elastin composite vascular scaffold (average diameter of 4.3±0.3 mm and length of 4.0±0.6 cm) was implanted first into either the left or right common carotid, determined randomly, and an equal length ePTFE control graft (GORE-TEX®, 4 mm diameter) was then implanted on the contralateral side. The study was continued for six hours or until both of the grafts occluded as determined by the Doppler flowrate readings at which point both grafts were excised.

The excised grafts were opened longitudinally, photographed and examined grossly for evidence of thrombi. Grafts were then cross-sectioned at 5 mm intervals. Alternate sections were snap frozen for cryoembedding, fixed in 10% neutral buffered formalin for paraffin embedding, or fixed in 2.5% glutaraldehyde for SEM analysis. Tissue was processed for histological staining or electron microscopy as described above to evaluate thrombosis on the surface and cell infiltration into the elastin composite vascular scaffold.

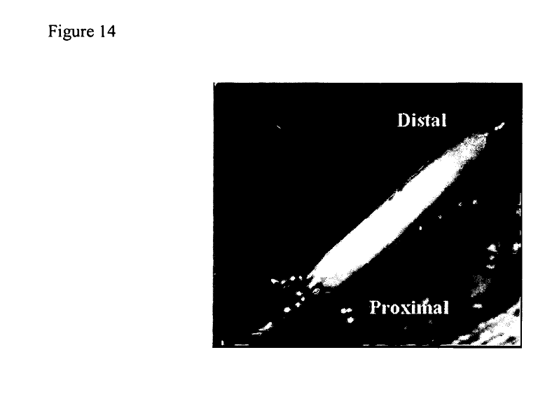

All six elastin composite vascular scaffolds were successfully sutured as interpositional grafts in the carotid artery of domestic swine with no significant difference in crossclamp times compared to the ePTFE controls (32±12 minutes vs 25±7 minutes, p=0.17, Paired t-test). Gross images, FIG. 14, and angiography indicated minimal size mismatch with the native artery. The bar indicates 6 mm. The implanted composite scaffolds displayed physiological pulses similar to the native artery as noted through visual observations.

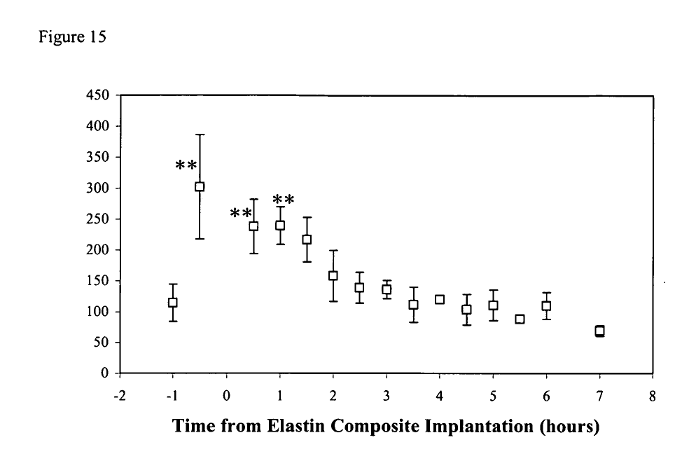

This domestic swine implantation model represents an aggressive thrombosis challenge; heparin is only administered during implantation, resulting in a transient increase of ACT times, which return to baseline ACT levels within 90 minutes, as shown in FIG. 15. FIG. 15 illustrates ACT as measured throughout the acute implantation. ACTs were taken prior to implantation (−60 minutes) and within an average of 30±11 minutes prior to elastin composite scaffold implantation (−30 minutes) and every 30 minutes throughout the procedure (average±standard deviation).

The times of the readings were normalized to the implantation time of the elastin composite scaffold and grouped in 30 minutes intervals. Only the −30, 30, and 60 minutes time points were significantly different from the pre-implant values, indicating that within 90 minutes of the composite scaffold implantation the heparin is rapidly metabolized by the swine (**p<0.01, ANOVA, Bonferroni).

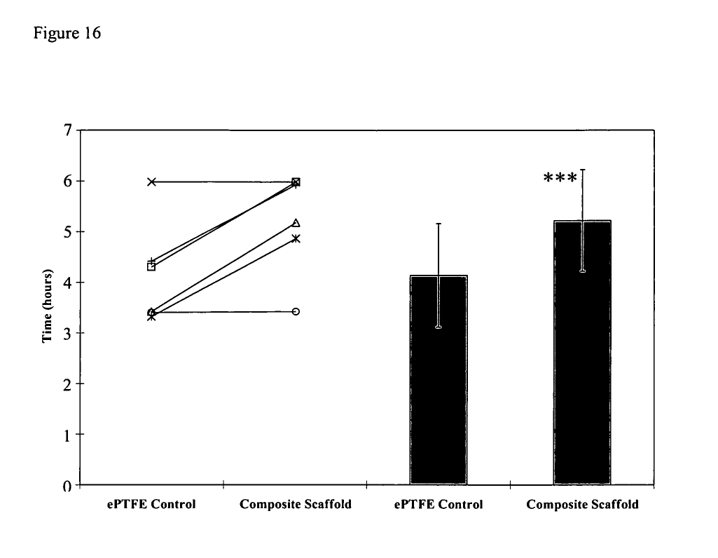

The elastin composite vascular scaffold always performed equal to or better than the ePTFE control graft during the six hour implant study, as shown in FIG. 16, a graph of occlusion times for individual experiments and the averages with six hours assigned to the patent vessels (average±standard deviation). Not only did the composite vascular scaffold have a better patency rate of 33% ( 2/6) compared to 16.5% (⅙) for the ePTFE control grafts, the average patency times were significantly longer for the composite vascular scaffolds, 5:14±1:00, compared to 4:09±1:01 for the ePTFE control grafts (FIG. 16, p<0.05, Paired t-test). The average patency times were determined by the Doppler flow measurements (and confirmed with angiography) with 6 hours used for the fully patent vessels.

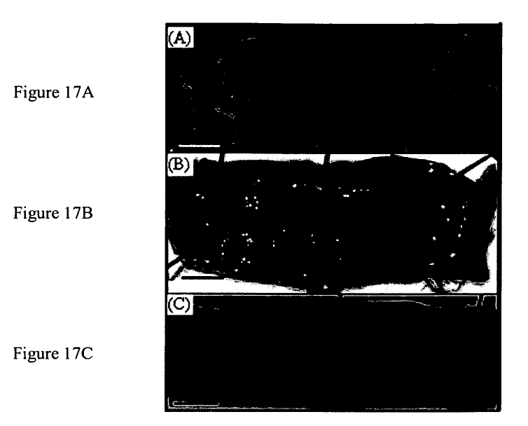

The gross images of representative explanted grafts, shown in FIGS. 17A-17C, demonstrate the range of reactions to the elastin composite scaffold with FIG. 17A, a patent elastin composite vascular scaffold, and 17B and 17C, occluded specimen of elastin composite vascular scaffold and ePTFE control graft, respectively, from the same animal. The thrombus in the elastin composite vascular scaffold, FIG. 17B, appears to be associated with the suture line, while it is throughout the ePTFE control graft, shown in FIG. 17C. The scale bars indicate 0.5 cm.

As shown in FIGS. 17A-17C, two of the six vessels had clean surfaces with no thrombus formation (FIG. 17A), while the remaining 4 scaffolds had isolated thrombi (FIG. 17B), which is most likely an injury response related to the suture line, rather than a reaction to the material. When clotted, the ePTFE control grafts contained pervasive thrombi (FIG. 17C).

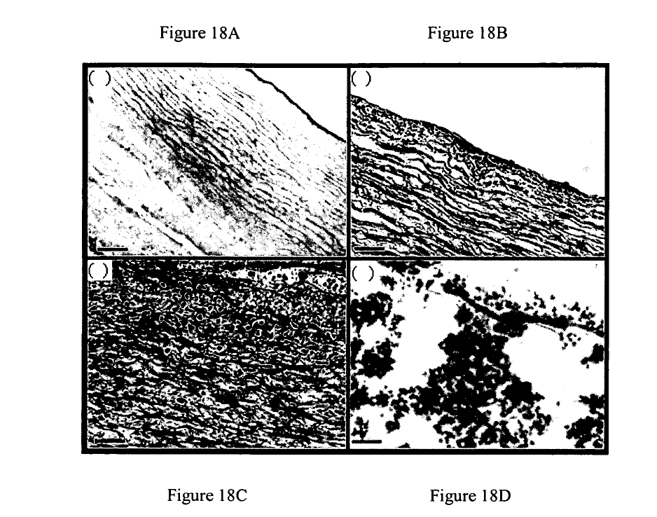

Histological images of the explanted grafts stained with Hematoxylin and Eosin are shown in FIGS. 18A-18D. In FIG. 18A, the scale bar indicates 100 μm. In FIGS. 18B, 18C, and 18D, the scale bars indicate 25 μm.

FIGS. 18A-18C indicate cell infiltration of varying degrees into the elastin composite scaffold. The ePTFE grafts, such as shown in FIG. 18D, were typically filled with red blood cells and minimal mononuclear cells. Cell infiltration into the elastin composite vascular scaffold varied from minimal cell infiltration in 18A, 18B, to maximal cell penetration depths of 130 microns in 18C. The animal with minimal cell infiltration into the elastin composite vascular scaffold 18A and 18B had an ePTFE control graft, 18D, filled with red blood cells throughout artery wall.

Using a purified elastin conduit as the basis of the graft allowed the formation of a complete arterial elastin matrix in which both the elastin content and fiber structure of a natural artery are restored. The ability of this elastin-based scaffold to store and return energy to the circulation was at least partially replicated in this graft, as evidenced by visual pulsation similar to that of a native artery both after implantation and during cyclic circumferential strain testing.

The porcine graft implantation animal model represents a robust thrombotic challenge with greater then 80% of clinically available ePTFE grafts occluding within six hours. This is likely due to the extensive arterial injury (arterial bisection and anastomosis) and for most animals, the use of a single heparin dose leading to a rapid return of ACTs to baseline levels within 90 minutes of implantation. One animal had an additional heparin dose to maintain the preset criteria of an ACT below 250 during the surgical implantation of both grafts.

Thus this model was designed not as a predictor of long term patency under optimal anticoagulant therapy, but rather as a direct test of acute thrombogenicity. In our model, there was no instance where the composite graft occluded first, and in five of the six animals the composite graft failed after the ePTFE control graft. There was one instance where the composite graft failed at the same time as the ePTFE control. Thus the elastin conduits displayed a significant increase in acute patency, suggesting that the flow surface is superior to clinically available ePTFE.

At least certain disclosed vascular grafts meet many of the design criteria for an ideal small diameter graft. The fabrication method is straightforward and may, in certain implementations, use only biologically sourced materials that are readily available. The manufacturing has been optimized to provide a consistent geometry using scaffold proteins to provide mechanical integrity naturally and, at least in certain implementations, without chemical cross-linking. Elastin as a blood-contacting surface is potentially less thrombogenic than other biologically sourced materials, such as collagen, or synthetic materials, such as PTFE or Dacron. Thus, the use of a composite graft having an elastin luminal layer may reduce or eliminate complications associated with tissue engineering techniques, such as cell seeding, or post implantation treatments, such as anticoagulant therapy.

Certain disclosed vascular grafts may be made reproducibly from naturally occurring protein matrices. The adhesion of the SIS to the elastin provides sufficient mechanical strength to withstand arterial pressures. The durability testing demonstrates that the adhesion between the layers is sufficient for short term experiments without delamination. The in vivo testing shows the graft to be suturable and patent over short periods of time. The ultimate strength of the composite graft far exceeds that of the elastin alone. The composite structure allows the thrombogenicity of the elastin scaffold to be studied in vivo.

It should be understood that the foregoing relates only to particular embodiments and that numerous modifications or alterations may be made without departing from the true scope and spirit of the invention as defined by the following claims.

We claim:

1. A composite multi-layered vascular graft prosthesis, comprising:
(A) a lumen-forming blood contacting inner layer consisting essentially of substantially acellular elastin; and
(B) an outer layer comprising a sufficient amount of a collagen matrix adhered to the inner layer to provide mechanical strength to the inner layer of elastin when the prosthesis is implanted in the body.

2. The graft prosthesis of claim 1, wherein the outer layer comprises substantially acellular collagen.

3. The graft prosthesis of claim 2, wherein the outer layer comprises acellular submucosa.

4. The graft prosthesis of claim 3, wherein the acellular submucosa is small intestinal submucosa.

5. The graft prosthesis of claim 1, wherein the inner layer consists essentially of elastin obtained from a blood vessel.

6. The graft prosthesis of claim 5, wherein the luminal layer of acellular elastin is obtained by chemical treatment of a blood vessel having elastin in its wall.

7. The graft prosthesis of claim 1, wherein the outer layer is adhered to the luminal layer by an adhesive comprising fibrin.

8. The graft prosthesis of claim 1, further comprising one or more growth factors in the prosthesis.

9. The graft prosthesis of claim 8, further comprising one or more growth factors in the adhesive.

10. The graft prosthesis of claim 1, wherein the luminal layer is a layer of substantially pure acellular elastin.

11. The graft prosthesis of claim 1, wherein the prosthesis is a tubular member having ends of a suitable size to be anastomosed at each of its ends to a blood vessel to establish patent flow through the blood vessel and prosthesis.

12. A method of performing a vascular anastomosis, comprising:
removing a segment of vasculature to provide an anastomotic end of a blood vessel;
placing the composite multi-layered graft prosthesis of claim 1 proximate the anastomtic end of the blood vessel; and
anastomosing the graft prosthesis to the anastomotic end of the blood vessel to establish patent flow through the graft prosthesis and vasculature.

13. The method of claim 12, wherein the step of anastomosing the graft prosthesis to the end of the blood vessel comprises suturing the graft prosthesis to the blood vessel.

14. The method of claim 12, wherein the outer layer of the graft prosthesis is adhered to the luminal layer by an adhesive.

15. The method of claim 12, wherein the elastin of the luminal layer is an elastin matrix isolated from the tissue of an organism.

16. A method of constructing a graft prosthesis comprising wrapping a collagen matrix around an elastin matrix to form a tubular graft prosthesis having a luminal blood-contacting layer consisting essentially of elastin, and an outer collagen layer that imparts mechanical strength to the graft prosthesis.

17. The method of claim 16, wherein at least one of the collagen matrix and the elastin matrix comprises a fibrillic matrix isolated from the tissue of an organism.

18. The method of claim 16, further comprising removing cells from the collagen matrix.

19. The method of claim 16, further comprising applying an adhesive to at least one of the collagen layer and the elastin layer to adhere the collagen and elastin layer to one another.

20. The method of claim 19, further comprising adding a growth factor to the adhesive.
